(19) United States
(12) Patent Application Publication
(10) Pub. No.: US 2009/0221921 A1
(43) Pub. Date: Sep. 3, 2009

(54) SIDE-FIRING LINEAR FIBER OPTIC ARRAY FOR INTERSTITIAL OPTICAL THERAPY AND MONITORING USING COMPACT HELICAL GEOMETRY

(76) Inventors: William J. Cottrell, Somerville, MA (US); Thomas Foster, Rochester, NY (US)

Correspondence Address: BLANK ROME LLP, WATERGATE, 600 NEW HAMPSHIRE AVENUE, N.W., WASHINGTON, DC, 20037, US

(21) Appl. No.: 12/296,886

(22) PCT Filed: Apr. 9, 2007
(86) PCT No.: PCT/US07/08870

§ 371 Date: Apr. 24, 2009

Related U.S. Application Data
(60) Provisional application No. 60/790,540, filed on Apr. 10, 2006.

(30) Foreign Application Priority Data
Apr. 6, 2007 (US) 11783199

(51) Int. Cl. A61B 6/00; A61B 18/22; A61N 5/067

(52) U.S. Cl. 600/478; 606/15

(57) ABSTRACT

An optical probe has multiple side-firing optical fibers which terminate in a linearly staggered fashion. A central fiber can be used as well. In diagnostic techniques, one fiber can be used as an emitter, while the others are used as receivers, or various fibers can be used as emitters and receivers at different times to form a map of the area. In therapeutic techniques, the treatment light can be emitted from the fibers in parallel or in sequence, and the fluence can be independently adjusted for each of the fibers.

### REFERENCE TO RELATED APPLICATIONS

The present application claims the benefit of U.S. Provisional Application No. 60/790,540, filed Apr. 10, 2006. Related information is disclosed in WO 2006/025940 A2, A3. The disclosures of both of the above-cited applications are hereby incorporated by reference in their entireties into the present disclosure.

### STATEMENT OF GOVERNMENT INTEREST

The work leading to the present invention was funded by NIH Grants P01CA55719, R01CA68409, and T32HL66988. The government has certain rights in the invention.

### FIELD OF THE INVENTION

The present invention is directed to an optic array for tissue measurements and other optical inspection and more particularly to such an optic array in which side-firing optical fibers terminate in a linearly staggered fashion.

### DESCRIPTION OF RELATED ART

The accurate, real-time determination of measurable quantities that influence or report therapeutic dose delivered by photodynamic therapy (PDT) is an area of active research and clinical importance. Photosensitizer evolution, including photobleaching and photoproduct formation, and accumulation of endogenous porphyrins provide attractive implicit dose metrics, as these processes are mediated by similar photochemistry as dose deposition and report cellular damage, respectively. Reflectance spectroscopy can similarly report blood volume and hemoglobin oxygen saturation.

Photodynamic therapy is a burgeoning cancer treatment modality in which a combination of light and drug is used to kill tumor cells with high selectivity. Leveraged with success in dermatology, ophthalmology, and directly accessible tissues, PDT is being expanded into treatment of prostate cancer, lung cancer, liver cancer, nodular basal cell carcinoma, and other interstitial applications. In order to deliver and monitor effective dose in these new applications, however, it is important to understand the optical properties of the tissue, which are often heterogeneous between applications and can even change during therapy. It is therefore important to make measurements before and during a treatment to plan the therapy and assess its progress.

### SUMMARY OF THE INVENTION

It is therefore an object of the invention to measure the optical properties of tissue.

It is another object of the invention to be able to do so over time.

It is another object of the invention to provide a device for characterization and quantification of chromatophores and fluorophores within turbid media.

It is another object of the invention to allow photodynamic therapy treatment source delivery and fluorescence and reflectance spectroscopies in needle- and catheter-accessible tissues.

To achieve the above and other objects, the present invention is directed to an optical probe having multiple side-firing optical fibers which terminate in a linearly staggered fashion as well as to an instrument incorporating such a probe. A central fiber can be used as well, and the fibers can be disposed in a catheter or needle. The fibers can be used in various ways. For instance, in diagnostic techniques, one can be used as an emitter, while the others are used as receivers, or various fibers can be used as emitters and receivers at different times to form a map of the area. In therapeutic techniques, the treatment light can be emitted from the fibers in parallel or in sequence, and the fluence can be independently adjusted for each of the fibers. In a combined therapeutic and diagnostic/monitoring technique, treatment light may be delivered through the central diffuser fiber while the side-firing fibers monitor fluence. Or, the treatment light administered through the diffuser may be gated off for a brief interval while the side-firing fibers are used for reflectance and/or fluorescence spectroscopy of the tissue volume.

### BRIEF DESCRIPTION OF THE DRAWINGS

Preferred embodiments of the present invention will be set forth in detail with reference to the drawings, in which:

FIGS. 1A and 1B show the construction of the probe according to a first preferred embodiment;

FIGS. 2A and 2B show an instrument incorporating the probe of FIGS. 1A and 1B and its use;

FIG. 3 shows a first use of the probe;

FIGS. 4A-4D show a second use of the probe;

FIG. 5 shows a third use of the probe;

FIG. 6 shows a fourth use of the probe;

FIG. 7 shows a modification of the probe for a fifth use; and

FIG. 8 shows a second preferred embodiment of the probe.

FIGS. 9A and 9B show a third preferred embodiment of the probe.

FIGS. 10A and 10B show a fourth preferred embodiment of the probe.

### DETAILED DESCRIPTION OF THE PREFERRED EMBODIMENTS

Preferred embodiments of the invention will bet set forth in detail with reference to the drawings, in which like reference numerals refer to like elements throughout.

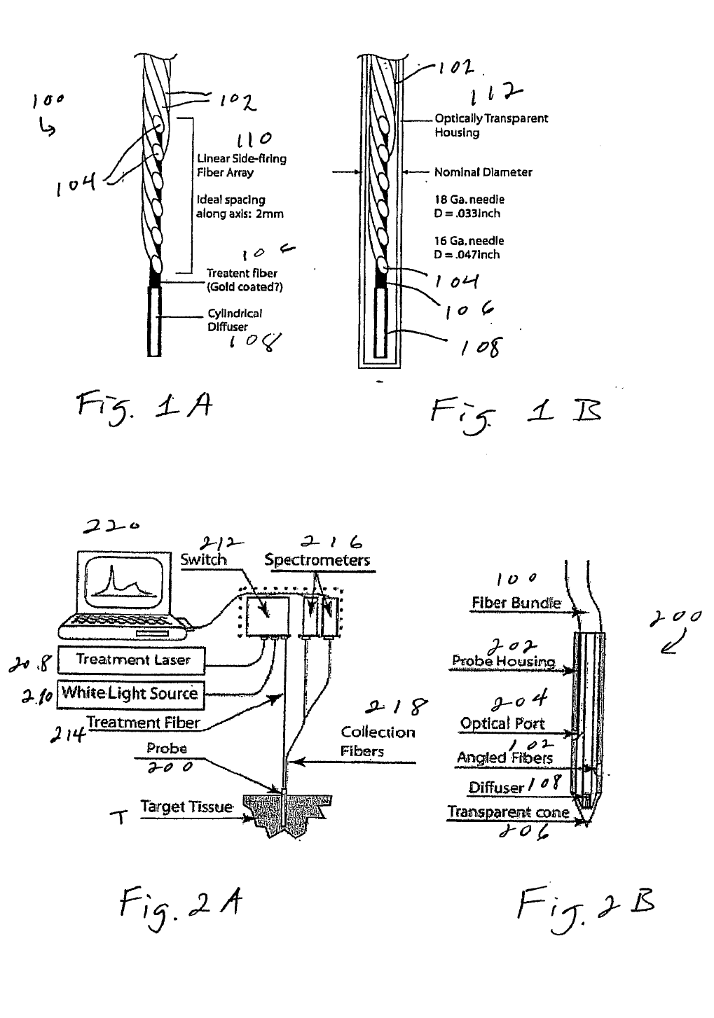

In a first preferred embodiment, as shown in FIG. 1A, the probe 100 includes seven optical fibers in the known “six-around-one” fiber bundle geometry. That geometry, while generally known in the art, is novel in the context of the present invention. Six fibers 102 are helically wound and terminate in fiber ends 104. A short segment of the central fiber 106 is coated with gold or another appropriate marker, allowing for x-ray guided positioning through a needle- or catheter-based delivery system, and is terminated with a cylindrical diffusing tip 108. Coatings other than gold, which are well known in the field, can be used in addition to, or instead of gold to render the device detectable by other imaging modalities, such as magnetic resonance or ultrasound.

The six outside fibers 102 are side-firing fibers, which are twisted around the central fiber 106 so that they form a linear array 110 along the long axis of the bundle. The ideal spacing along the axis, in the present embodiment, is 2 mm. By arranging the fibers in that manner, the probe is optimized for compactness, while providing a linear array of fiber ends. As shown in FIG. 1B, the entire bundle can be encased in a transparent capillary 112 which can be inserted into tissue through a catheter or needle. Exemplary nominal diameters of the capillary are 0.033 inch for insertion into an 18-gauge needle and 0.047 inch for insertion into a 16-gauge needle.

The probe can be inserted into any needle- or catheter-accessible tissue via standard methods and guided with x-ray or other imaging or guidance. The probe is useful in planning, delivering and monitoring PDT in accessible tissues. As shown in FIGS. 2A and 2B, a probe assembly 200 is formed by inserting the above-described probe 100 into a needle or probe housing 202 having optical ports 204 corresponding to the ends 104 of the fibers 102 and a transparent cone 206 corresponding to the diffuser 106. The probe assembly 200 is connected to a treatment laser 208 and a white-light source 210 through a switch 212 and a treatment fiber 214 and to spectrometers 216 through collection fibers 218. A computing device 220 analyzes the outputs of the spectrometers 216. The probe assembly is shown as being inserted into tissue T.

Before treatment, for example, white light reflectance spectroscopy can be used to assess the optical properties of the tissue in which the probe is located. This can be used to determine the scattering and absorption coefficients of the tissue, which can be used to determine the amount and distribution of photosensitizer present and the volume and oxygenation of hemoglobin. Those parameters are useful for planning a PDT treatment. White light spectroscopy can nominally be performed by using one of the fibers in the linear array as a source by directing broadband light through that fiber. Spectra can then be collected from the other fibers, and a fitting algorithm can be used with the data to determine the optical properties of the tissue.

During treatment, either one of the side-firing fibers or the cylindrical diffusion fiber can act as a source, while the other fibers collect fluorescent spectra concurrently. That provides information on dose metrics such as fluorescence photobleaching and photoproduct accumulation. Additionally, brief treatment interruptions can be used to interrogate the tissue with white light in order to monitor changes in blood volume and blood oxygenation.

The optical probe could be integrated into a portable PDT system straightforwardly. For example, its design is compatible with the instrument disclosed and claimed in the above-cited PCT publication.

The probe described above can be used in many ways, including the following.

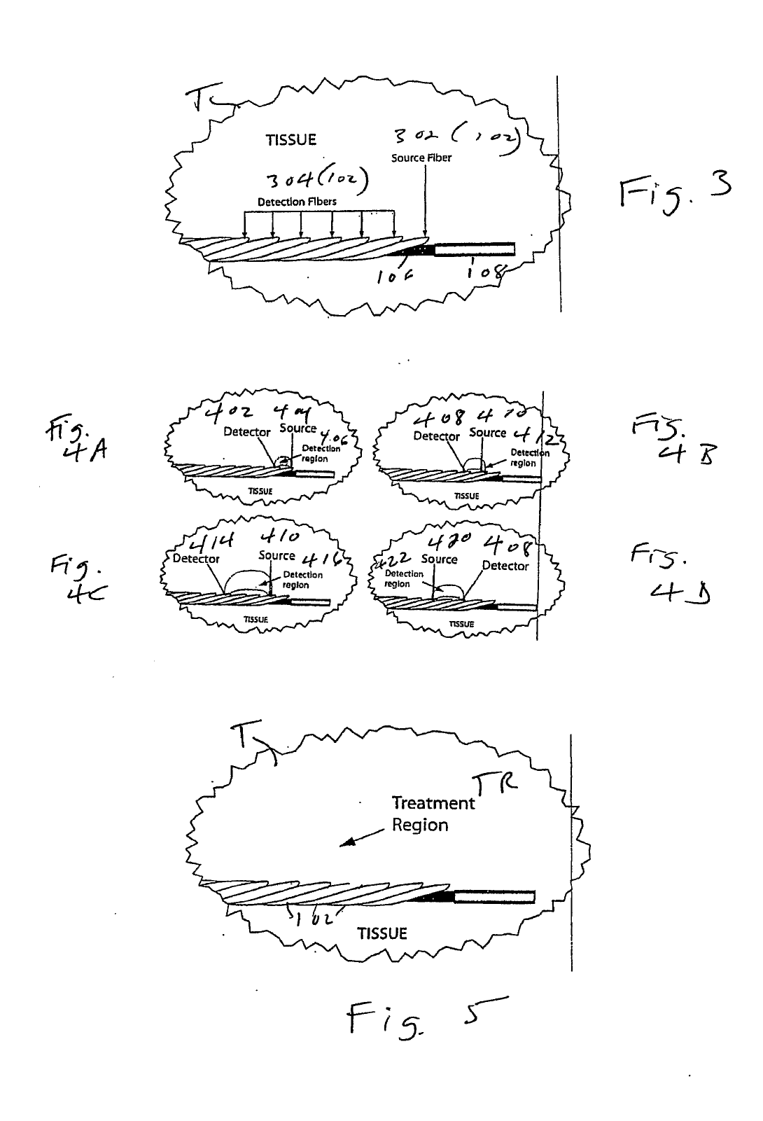

Single treatment/interrogation beam with many simultaneous data collection fibers, constituting a linear detection array: This functionality is described above and is likely the most immediate use for the probe. As shown in FIG. 3, a single side-firing fiber 102 functions as the source fiber 302, while the remaining side-firing fibers 102 function as detection fibers 304.

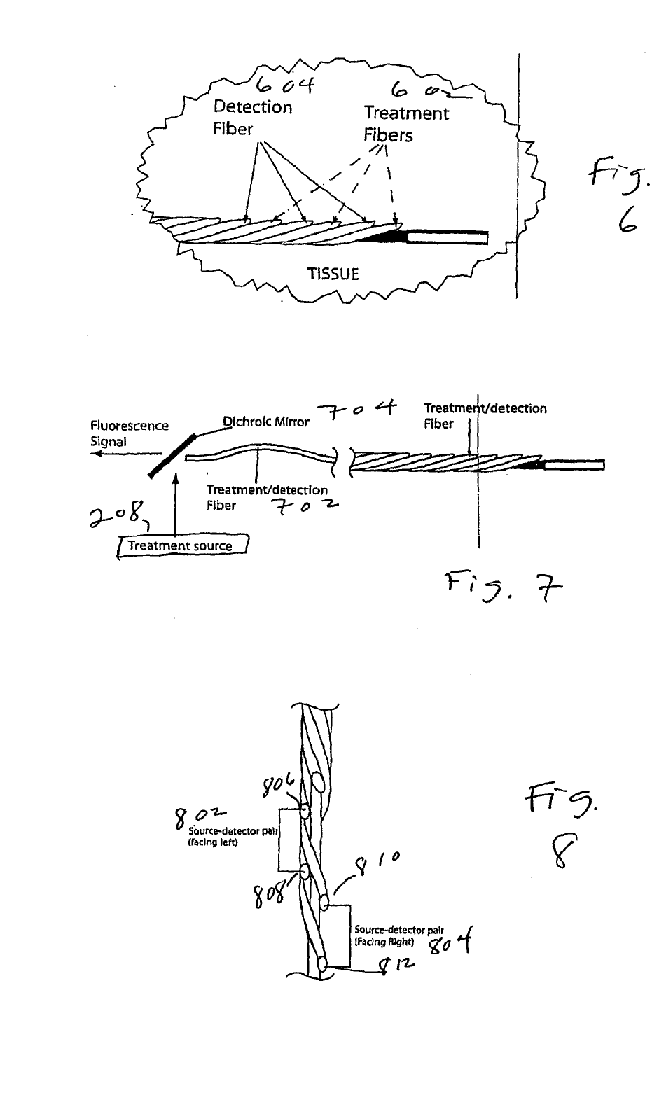

Multiple interrogation beams with multiple detectors: Several fibers can be used to perform optical interrogation using fluorescence or reflectance spectroscopy. For example, as shown in FIG. 4A, a first fiber can be used as a white light source 404, and a second adjacent fiber 402 can be used for detection, creating a detection region 406. Then, as shown in FIG. 4B, the second fiber can be used as a source 410, and a third fiber can be used as a detector 408, creating a detection region 412. As shown in FIG. 4C, the same source 410 can be used with a different detector 414 to create a detection region 416. As shown in FIG. 4D, the same detector 408 as in FIG. 4B can be used with a source 420 to create a detection region 422. Different source/detector fiber combinations with appropriate optical switching can be used to map out local volumes within the tissue along the axis of the probe.

Multiple treatment beams with independently adjustable fluorescence rates: As shown in FIG. 5, each optical fiber 102 can be used to deliver the PDT treatment beam to a treatment region TR in the tissue T. Delivery of PDT could be done serially (cycling through the fibers) or in parallel (all fibers being used concurrently). The fluence rate of light delivered through each fiber can be optimized independently so that an optimal light distribution in the tissue can be obtained. That method could make use of the multiple interrogation method described above and use the map of local regions to determined an optimal fluence rate for each delivery fiber.

Multiple treatment beams with multiple simultaneous detection: As shown in FIG. 6, first plurality of fibers 602 is used to deliver the PDT treatment beam, and a second plurality of fibers 604 is used for detection. The fluence rate of light delivered through each fiber can be optimized independently, so that an optimal light distribution in the tissue can be obtained. That method could make use of detector feedback to determine an optimum fluence rate for each delivery fiber.

Multiple treatment beams with fluorescence detection/feedback: Each optical fiber can be used to deliver the PDT treatment beam. Fluorescence spectra are collected during PDT delivery through either adjacent dedicated detection fibers or backwards through the delivery fiber. Detected signals can be used as feedback to control therapy delivery. FIG. 7 shows a treatment/detection fiber 702 and a dichroic beamsplitter 704 used at the distal (non-probe) end of the fiber.

Variations of the probe geometry described above can also be realized. For example, as shown in FIG. 8, pairs 802, 804 of fibers can be used, in which one fiber 806, 810 serves as a source and the other fiber 808, 812, as a detector. Tissue optical properties and/or treatment can be made around the probe.

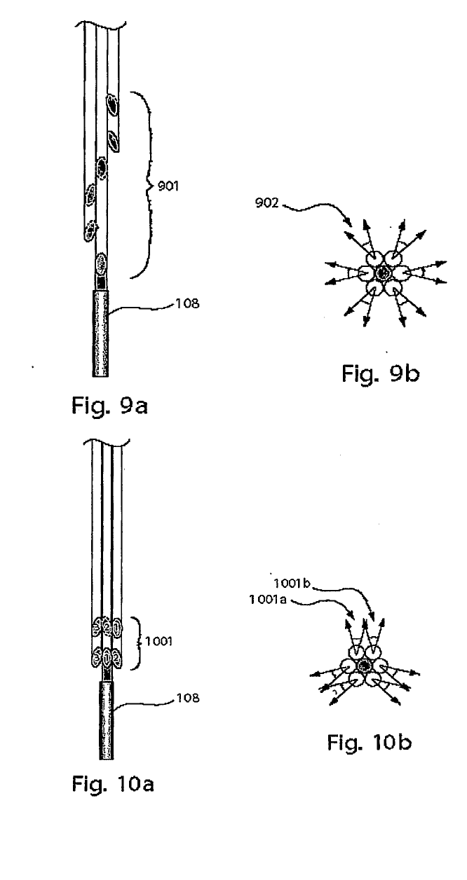

Another geometry uses fibers which are staggered in axial position and direction so that they form a “spiral staircase” structure as shown in FIG. 9A. In this embodiment, cylindrical diffuser 108 is surrounded by side-firing fiber array 901. Each fiber in the array is offset linearly from the adjacent fibers along the axis of the probe. Axial view FIG. 9B illustrates the 6-around-1 probe geometry and the acceptance/delivery cone 902 for the light entering/exiting one fiber.

Yet another geometry uses fibers pairs in which one fiber in the pair is offset in axial position, and both fibers face the same direction as shown in FIG. 10. In this embodiment, cylindrical diffuser 108 is surrounded by side-firing fiber array 1001. Three fiber pairs are arranged in the probe such that each pair has one fiber substantially at the same first location along the axis of the probe and a second fiber substantially at the same second location along the axis of the probe, as shown in FIG. 10A. Axial view FIG. 10B illustrates the 6-around-1 probe geometry and the acceptance/delivery cones 1002a and 1002b for the light entering/exiting the fiber in one fiber pair.

While preferred embodiments of the invention have been set forth above, those skilled in the art who have reviewed the present disclosure will readily appreciate that other embodiments can be realized within the scope of the invention. For example, numerical values are illustrative rather than limiting. Therefore, the invention should be construed as limited only by the appended claims.

We claim:

1. An optical fiber probe comprising:
a plurality of helically wound side-firing optical fibers; and
a plurality of fiber ends, one on each of the fibers, the fiber ends being arranged in a linear side-firing array.

2. The optical fiber probe of claim 1, further comprising a central optical fiber around which the plurality of fibers are wound.

3. The optical fiber probe of claim 2, further comprising a cylindrical diffuser on an end of the central optical fiber.

4. The optical fiber probe of claim 2, wherein the central optical fiber comprises a material which is opaque to an imaging modality.

5. The optical fiber probe of claim 4, wherein the material comprises gold.

6. The optical fiber probe of claim 1, further comprising a catheter in which the fibers are disposed.

7. The optical fiber probe of claim 1, further comprising a needle in which the fibers are disposed.

8. The optical fiber probe of claim 7, wherein the needle has optical ports corresponding to the fiber ends.

9. The optical fiber probe of claim 8, further comprising a central optical fiber around which the plurality of fibers are wound and a cylindrical diffuser on an end of the central optical fiber, and wherein the needle comprises a transparent cone at an end of the needle.

10. The optical fiber probe of claim 1, wherein the plurality of fibers are wound in a same direction.

11. The optical fiber probe of claim 1, wherein the plurality of fibers comprise pairs of fibers which are wound in opposite directions.

12. An optical fiber probe system comprising:
a plurality of helically wound side-firing optical fibers;
a plurality of fiber ends, one on each of the fibers, the fiber ends being arranged in a linear side-firing array;
at least one light source for outputting light from at least one of the fiber ends through at least one of the fibers; and
a spectrometer for receiving light from at least one of the fiber ends through at least one of the fibers and for analyzing the received light.

13. The system of claim 12, wherein the at least one light source comprises a source of white light.

14. The system of claim 12, wherein the at least one light source comprises a treatment laser.

15. The system of claim 12, wherein the fibers are connected to the at least one light source and the spectrometer such that the fibers can be selectively connected either to the at least one light source or to the spectrometer.

16. A method for treating or diagnosing tissue, the method comprising:
(a) inserting an optical fiber probe into the tissue, the optical fiber probe comprising a plurality of helically wound side-firing optical fibers and a plurality of fiber ends, one on each of the fibers, the fiber ends being arranged in a linear side-firing array; and
(b) applying light to the tissue through at least one of the fibers.

17. The method of claim 16, wherein the light is light from a treatment laser.

18. The method of claim 17, wherein the light is emitted from the plurality of fibers in parallel.

19. The method of claim 17, wherein the light is emitted from the plurality of fibers in sequence.

20. The method of claim 17, wherein the light is emitted from the plurality of fibers, and wherein a fluence of the light is independently adjusted for each of the fibers.

21. The method of claim 16, wherein the light is diagnostic light, and further comprising:
(c) receiving light from the tissue through at least one other one of the fibers; and
(d) spectroscopically analyzing the received light for diagnosis.

22. The method of claim 21, wherein the light received in step (c) is reflected light.

23. The method of claim 21, wherein the light received in step (c) is fluorescently emitted light.

24. The method of claim 21, wherein step (b) is performed through one of the fibers, and wherein step (c) is performed through other ones of the fibers.

25. The method of claim 21, wherein steps (b) and (c) are performed at different times using different ones of the fibers, and wherein step (d) is performed for different regions in the tissue.
